(19) United States
(12) Patent Application Publication
(10) Pub. No.: US 2023/0146726 A1
(43) Pub. Date: May 11, 2023

(54) STABLE TOCOTRIENOL WITH IMMUNOMODULATING ACTION

(71) Applicants: FONDAZIONE IRCCS ISTITUTO NAZIONALE DEI TUMORI (50%), Milano (IT); FAMAFARM S.R.L. (50%), Trento (IT)

(72) Inventors: Cristina Ferraris, Milano (MI) (IT); Francesca Ferri, Trento (IT); Maria Teresa Armano, Trento (IT)

(73) Assignees: FONDAZIONE IRCCS ISTITUTO NAZIONALE DEI TUMORI (50%), Milano (IT); FAMAFARM S.R.L. (50%), Trento (IT)

(21) Appl. No.: 17/906,477

(22) PCT Filed: Mar. 16, 2021
(86) PCT No.: PCT/IB2021/052163

§ 371 Date: Sep. 16, 2022

(30) Foreign Application Priority Data
Mar. 16, 2020 (IT) 102020000005575

(51) Int. Cl. A23B 9/08; A23L 3/44

(52) U.S. Cl. CPC A23B 9/08; A23L 3/44

(57) ABSTRACT

The present patent application relates to a lyophilisation process of Annatto Bixa Orellana L. powder having a tocotrienol content >35% (w/w), comprising a pre-freezing step and a freeze-drying step, in which the Annatto powder resulting from the freeze-drying step (c) has a tocotrienol content >39% (w/w). The invention also relates to pharmaceutical compositions comprising lyophilised

Annatto powder obtained with the process of the invention and therapeutic uses of such compositions, in particular to support cancer therapy.

### FIELD OF THE INVENTION

The present invention relates to a process for treating Annatto powder, the pharmaceutical compositions comprising the same and the therapeutic uses for which it is intended.

### BACKGROUND ART

Vitamin E, also known as Tocopherol, is a fat-soluble vitamin which mammals, including humans, introduce into the body through diet. It accumulates in the liver and therefore does not need to be taken daily. The body releases it in small doses when the use thereof becomes necessary.

Of all the vitamins, it is definitely the most widespread. The remarkable antioxidant and free radical counteracting properties thereof, as well as the ability thereof to promote cell renewal, have been scientifically demonstrated. These features make it an important tool for cancer prevention.

Vitamin E is sensitive to heat and light, so it tends to degrade in the presence of high temperatures or when exposed to light.

It is widespread in foods, especially in oily fruits (such as olives, peanuts, corn) and wheat seeds. It is also found in grains, nuts, and leafy green vegetables. The daily vitamin E requirement is around 8-10 mg.

Vitamin E, or tocopherol, deficiency can usually occur when malnutrition occurs, when consuming unbalanced or malabsorbing diets. In younger subjects, it may cause growth and development defects. In general, a lack of vitamin E may be at the base of the onset of nervous system disorders.

An excess of vitamin E, or tocopherol, is quite rare. When it occurs, it can have negative consequences due to the resulting increase in blood pressure, which can be dangerous for those already suffering from hypertension.

Excess vitamin E can also cause problems for those suffering from thyroid problems, as it can reduce the hormones in this gland. Other consequences of excess vitamin E can be general fatigue, digestive disorders, nausea, and vomiting.

Vitamin E has important anticancer functions by virtue of the powerful antioxidant action thereof which allows it to protect cell membranes and intervene directly on particular transcription factors used by cancer cells to proliferate. But this vitamin also plays an important role in relation to diseases of cardiovascular origin, being able to reduce the phenomena of platelet aggregation with consequent reduction of the formation of emboli, plaques, and thrombi (blood clots) in the arteries.

Vitamin E is also a valuable anticoagulant because it prevents excessive blood clotting without, however, preventing the normal coagulation required in the event of wounds, useful to stop bleeding. Finally, vitamin E reduces cardiovascular risk as, based on its activity, the level of so-called good cholesterol increases to the detriment of the “bad” cholesterol.

Biochemically, this vitamin actually consists of two subgroups of molecules, tocopherols and tocotrienols. Each of these two subgroups in turn is divided into four isoforms: alpha, beta, gamma, and delta.

When isolated, each of these isoforms retains the same virtues of the whole complex, in many cases the isolation of an isoform greatly amplifies the qualities and features thereof

Among these isoforms, delta-tocotrienol is probably the most studied in the context of oncological diseases. According to the scientific literature, tocotrienols have positive effects on various aspects of human health (anti-inflammatory action, prevention of cardiovascular and neurodegenerative diseases, etc.). Some in vitro and preclinical studies have shown that δ-TT (delta-tocotrienol) is the most active tocotrienol in counteracting the proliferation of human melanoma cells.

Tocotrienols are found in high concentrations in unrefined palm oil (50% of the vitamin E compound extract consists of δ-TT and γ-TT) but also and especially in Annatto seeds (Bixa Orellana; 99% δ-TT) (https://effegilab.com/delta-tocotrienolo-una-super-vitamina/).

In view of the multiple biological properties of tocotrienol, the ingredient is now used in the formulation of dietary supplements; these include Tocotrienol-FG, marketed by EFFEGILAB, which favourably acts on the control of carbohydrates and the protection of cells from oxidative stress.

The supplement (capsules) contains Annatto seed powder (Bixa orellana L.)—34.8%—with 35% tocotrienol content; the recommended daily dose is one capsule per day, where the Annatto content per daily dose is 155 mg.

Problems of the Prior Art

Annatto powder used as a raw material for the purposes of the invention is obtained by the process described in International Patent Application WO 00/71531, starting from Annatto oil, a by-product which is obtained by extracting the red/orange dye commonly known as annatto.

The annatto dye is obtained from the shelled seeds of Bixa Orellana L. by submitting them to an extraction which can be conducted in aqueous solution or in organic solvents.

In the case of extraction in aqueous solution, the seeds are immersed in an acid-based caustic solution (e.g., sulphuric acid); under such conditions, the dye precipitates, separating from the caustic aqueous phase, and can be filtered off. The Annatto oil is separated from the caustic aqueous phase by centrifugation or sedimentation.

In the case of solvent extraction, the seeds are immersed in an organic solvent solution (e.g., hexane, acetone, or alcohol); the mixture is allowed to cool so that the annatto precipitates and can be separated from the organic phase by filtration.

The Annatto oil thus obtained is then subjected to the process described in WO 00/71531 which, briefly, consists of a volatilisation of the residual solvent, regardless of whether it is the caustic aqueous solution or the organic solvent. For this purpose, a series of evaporators are used in series, which allow the distillation of the Annatto oil without the use of solvents, obtaining a concentrated tocotrienol product. The distillate can have a tocotrienol content of 20-90% by weight and a residual solvent content (whether caustic aqueous solution or organic solvent) of 0.05 to 0.5% by weight.

The tocotrienol distillate is then reduced to powder by mixing with tapioca dextrin and optionally silicon dioxide, thereby obtaining Annatto powder preferably used as a raw material for the purposes of the invention.

The Annatto powder to be subjected to the process of the invention (content in tocotrienols >35% by weight) has
poor water solubility (1 mg/ml) responsible, in vivo, for a low bioavailability of the active ingredients;
a high concentration of water (about 5% water) which limits the stability thereof over time;
a bulk density of 0.55 to 0.60 g/cm³.

In therapeutic scenarios other than dietary supplementation, where it is necessary to achieve daily Annatto doses above 155 mg/day, the aforementioned problems make it difficult to develop therapeutic plans to which the patient can easily adhere.

The amount of Annatto powder which would be necessary for the formulation of a higher dose administration unit, in combination with the appropriate excipients, would be such as to require the formulation of pharmaceutical forms (capsules/tablets) which are too large to swallow or, if of standard size, characterised by an administration frequency which is too high to ensure good patient compliance.

### SUMMARY OF THE INVENTION

From preliminary results of a clinical study and subsequent results, the Applicants have observed that tocotrienol from Annatto, administered at a daily dose of at least 400 mg/day, exhibits not only an antioxidant and anti-inflammatory effect, but also a selective antagonism in the neoplastic sense, in the immunological profile, in patients with non-metastatic breast cancer.

Noting the surprising activities of tocotrienol at such a dose, the Applicants found that by treating Annatto powder with a dedicated lyophilisation process it is possible to obtain a powder with improved stability, solubility, and bioavailability, which can be more easily formulated into oral dosage forms.

The object of the present invention is therefore the lyophilisation process of Annatto powder, the pharmaceutical compositions containing the lyophilised powder and the use thereof as a medicine, preferably in the oncological treatment of non-metastatic breast cancer in the pre-operative stage or in addition to the adjuvant or neo-adjuvant treatment of a systemic or regional type.

Advantages of the Invention

The studies conducted by the Applicants have shown that tocotrienol from Annatto can be used, in the pre-operative stage, for the treatment of non-metastatic breast cancer, as well as a generic antioxidant/anti-inflammatory/immunomodulatory agent; based on the information available to the Applicants, such therapeutic use is unprecedented in the literature.

Furthermore, the lyophilisation process allows the water content of the extract to be reduced by a process which does not use heat or extraction additives.

By reducing the hygroscopy of the raw material, a powder with lower volume is advantageously obtained which can be formulated in administration units suitable for oral assumption; the powder can then be conveyed in standard size pharmaceutical forms, in sufficient quantities to ensure that the desired daily dose is achieved.

It is therefore possible to develop a treatment plan with a maximum of two administrations per day, with greater compliance by the patient. It should be noted that, in order to reach the dose necessary for adjuvant oncology treatment with the formulations currently available on the market, it would have been necessary to administer eight capsules/day.

It should be noted that such results are not obtainable by employing generic lyophilisation techniques: as evidenced by Comparison example No. 2, by applying a standard lyophilisation process to the raw material (Annatto powder−tocotrienols≥35%), characterised by a pre-freezing, a main freeze-drying step and a secondary freeze-drying step, an unsatisfactory powder for the formulation purposes of the invention is obtained, since it is sticky and with an excessive apparent volume.

The lyophilisation treatment, in addition to providing advantages from a technological point of view, improved the solubility of the Annatto powder (increase of about 20%), the bioavailability thereof and, therefore, the in vivo effectiveness of the active ingredient. In this regard, reference is made to the section of the application dedicated to the experimental data to understand in detail the biological advantages which emerged from the use of the lyophilised Annatto powder.

### DETAILED DESCRIPTION OF THE INVENTION

Bixa Orellana L. is a plant native to tropical America (Mexico, Belize, Costa Rica, El Salvador, Guatemala, Honduras, Nicaragua, Panama, Colombia, Venezuela, Guyana, French Guiana, Suriname, Ecuador, Peru, Brazil, Bolivia, Paraguay, and Argentina). The fruits are ovoid berries with a sharp apex, about 35 mm long, generally bright red, densely covered with bristles 8 mm long and containing many small angular seeds, about 4 mm long, whose coating (aryl), rich in apo-carotenoids and carotenoids, gives rise to the dye commonly known as annatto (https ://www. m onaconatureency cl op edi a. com/bixa-orellana/).

For the purposes of the present invention, Annatto powder (pre-lyophilisation) means the powder obtainable by treatment of the seeds of Bixa Orellana L. As previously described, the Annatto powder (pre-lyophilisation) preferred for the purpose of applying the method of the invention is a mixture comprising Annatto oil (50-55%, tocotrienol content ≥35%), preferably tapioca dextrin (45-48%) and optionally silicon dioxide (1-2%). Such Annatto powder preferably contains a water residue between 0.05-0.5% by weight.

It should be noted that the Annatto powder used for the purposes of the present invention (pre-lyophilisation Annatto powder) is known to those skilled in the art and is commonly available on the market (such as, for example, supplied by Nutraceutica S.r.l.).

The lyophilisation process according to the invention comprises the steps of:

a) preparing Annatto powder having a tocotrienol content ≥35% (w/w);

b) pre-freezing the Annatto powder at a temperature lower than −15° C.;

c) submitting the pre-frozen powder according to step (b) to a freeze-drying process, characterised in that the freeze-drying step (c) comprises the sub-steps of

i) performing a first drying at a pressure less than 2.5 mbar and at a temperature between −45° C. and 0° C.;

ii) performing a second drying, following the first drying (i), at a constant pressure greater than the pressure of the first drying, at a temperature between 0° C. and 20° C., in which the Annatto powder resulting from the freeze-drying step (c) has a tocotrienol content ≥39% (w/w).

Preferably, the Annatto powder resulting from the freeze-drying step (c) (also referred to herein as lyophilised powder) has a tocotrienol content between 39% and 46% (w/w), preferably between 43% and 46% (w/w), preferably equal to 43%, 44%, 45%, 46%, 47%, 48%, 49%, 50% (w/w).

It should be noted that the tocotrienol from Annatto is a mixture of the alpha, beta, gamma and delta isoforms of tocotrienol. Preferably, the tocotrienol from Annatto is a mixture of alpha and delta tocotrienol. Preferably, the alpha and delta tocotrienol are in a weight ratio of 1:99 with each other.

Preferably, the Annatto powder to be subjected to the lyophilisation process has a tocotrienol content between 35% and 42% (350-420 mg/g), preferably between 39% and 42%, preferably equal to 39%, 40%, 41%. It should be noted that the lyophilisation process according to the present invention allows to reduce the weight of the pre-lyophilisation Annatto powder; such a reduction is attributable to the loss of residual water. Preferably, the lyophilisation process allows to reduce the weight of the Annatto powder by about 1.5% (w/w), preferably to reduce the Annatto powder weight by an amount between 1.5% and 5%, preferably between 3% and 5%. Therefore, the lyophilised Annatto powder has a residual water content of less than about 4.0% (w/w), preferably less than about 3.5% (w/w), preferably less than 3%, preferably between 3% and 2% (w/w).

It should be noted that the lyophilisation process according to the present invention allows to increase the solubility of the Annatto powder by about 20% with respect to the initial value (pre-lyophilisation).

According to a preferred embodiment, the pre-freezing step (b) is performed at temperatures between −20° C. and −40° C., preferably −30° C. and −40° C., preferably between −35° C. and −40° C., preferably −35° C., −36° C., −37° C., −38° C., −39° C., −40° C.

It should be noted that according to a preferred embodiment, the Annatto powder is ground prior to being pre-frozen according to step (b) of the method.

Preferably, the pre-freezing step (b) lasts longer than 9 hours, preferably between 12 and 20 hours, preferably between 15 and 20 hours, preferably 15, 16, 17, 18, 19, 20 hours.

Preferably, the pre-freezing step (b) is performed at a rate between 2.0° C/h and 2.2° C/h.

Preferably, the pre-freezing step (b) is carried out in bulk in external pre-freezers (or refrigerator cells); preferably, the pre-freezing step (b) is static.

According to a preferred embodiment, the first drying step (i) lasts more than 30 hours, preferably between 30 and 37 hours, preferably 30, 30.5, 31, 31.5, 32, 32.5, 33, 33.5, 34, 34.5, 35, 35.5, 36, 36.5, 37 hours; preferably, the first drying step (s) is performed in a plurality of steps (sub-steps), each step being characterised by a duration between 0.5 and 4 hours.

Preferably, the temperature decreases at a rate of 0.5° C/step during the first drying (i).

According to a preferred embodiment, the first drying (i) is performed at a pressure <2.5 mbar, preferably between 2.0 mbar and 0 mbar, preferably between 1.5 mbar and 0.2 mbar, preferably between 0 and 1.2 mbar.

According to a preferred embodiment, the pressure decreases at a rate between 0.02 mbar/hour and 0.06 mbar/hour, preferably between 0.03 mbar/hour and 0.05 mbar/hour during the first drying (i).

According to a preferred embodiment, the second drying (ii) lasts at least 12 hours, preferably between 12 and 15 hours, preferably equal to 12, 13, 14, 15 hours. Preferably, the second drying (ii) is performed in a plurality of steps (sub-steps), each step being characterised by a duration between 1 hour and 4 hours.

Preferably, the temperature increases at a rate of 5° C/step during the second drying.

Preferably, the second drying (ii) is performed at atmospheric pressure; preferably, the second drying (ii) is performed at a pressure between 900 mbar and 1000 mbar.

It should be noted that the second drying (ii) is performed at a constant pressure over time.

It should be noted that, according to the preferred embodiment, both the first drying (i) and the second drying (ii) are performed in bulk, directly on the freeze-drying trays of the lyophiliser.

According to a preferred embodiment, the Annatto powder to be submitted to lyophilisation is distributed in the lyophiliser trays at a thickness not exceeding 2 cm.

It should be noted that according to a preferred embodiment, the Annatto powder is prepared, in step (a) of the process, in combination with a cryo-preservative.

Preferably, said cryo-preservative is selected from the group consisting of: Glycine, Alanine, Leucine, Tryptophan, and combinations of the foregoing.

Preferably, such a cryo-preservative is Glycine.

A further object of the present invention is a pharmaceutical composition comprising the lyophilised powder of Annatto Bixa Orellana L. obtained by the process described above, in combination with suitable excipients and/or diluents, in which the Annatto powder has a tocotrienol content >39% (w/w).

The excipients and/or diluents are selected from those known in the state of the art for making solid pharmaceutical forms for oral administration, such as adsorbent agents, bulking agents, lubricants, anti-adhesives.

It should be noted that, as a result of the lyophilisation, the pharmaceutical composition preferably comprises one or more cryo-preservatives, useful for protecting the active ingredient during the freeze-drying steps under extreme pressure and temperature conditions.

Preferably, the lyophilised Annatto powder has a tocotrienol content between 39 and 46% (w/w).

Preferably, the pharmaceutical composition according to the present invention is intended for use as a medicine (for therapeutic use).

Preferably, the composition of the invention is used in the oncological treatment of non-metastatic breast cancer, in the pre-operative stage.

Preferably, the composition of the invention is used to support (in addition to) the primary oncological treatment of solid tumours.

Preferably, the composition of the invention is used to support (in addition to) systemic or regional adjuvant or neo-adjuvant therapy for the cancer treatment of subjects with non-metastatic breast cancer.

It should be noted that adjuvant refers to the medical treatment applied after surgery or radiation therapy has achieved a radicality target on the tumour.

It should be noted that neo-adjuvant refers to the medical treatment applied before loco-regional therapy (surgery or radiation therapy), to reduce the size of the tumour and facilitate the removal thereof.

It should be noted that systemic adjuvant or neo-adjuvant treatment preferably refers to a chemotherapeutic or hormonal oncology treatment (hormone therapy).

It should be noted that adjuvant or regional neo-adjuvant treatment preferably refers to radiotherapy treatment.

It should be noted that the use of the composition of the invention to support (in addition to) adjuvant or neo-adjuvant oncology therapy refers both to the simultaneous combined use of tocotrienol from Annatto and systemic or regional therapy (e.g., both tocotrienol and neo-adjuvant used prior to surgery or radiation therapy); and to the non-simultaneous combined use of tocotrienol from Annatto and systemic or regional therapy (e.g., tocotrienol from Annatto in the pre-operative stage and, following surgery or radiation therapy, the application of systemic or regional adjuvant treatment).

According to a preferred embodiment, the composition of the invention is administered at a therapeutic dose of at least 400 mg/day, preferably between 400 mg/day and 800 mg/day, preferably between 400 mg/day and 600 mg/day, preferably 400 mg/day, 450 mg/day, 500 mg/day, 550 mg/day, 600 mg/day.

Preferably, the composition of the invention, for the therapeutic uses indicated above, comprises Annatto powder in an amount between 60% and 90%, preferably between 70% and 90% (w/w), preferably between 80% and 87% (w/w), preferably equal to 81%, 82%, 83%, 84%, 85%, 86%, 87%.

Preferably, the composition of the invention further comprises a cryo-preservative in an amount between 0.5% and 5% (w/w), preferably between 1% and 3.0% (w/w), preferably between 2.0 and 3.0% (w/w), preferably 2.1%, 2.2%, 2.3%, 2.4%, 2.5%, 2.6%, 2.7%, 2.8%, 2.9%. As described above regarding the cryo-preservative, it applies in this case as is.

The composition obtained by the method of the invention can advantageously be conveyed in standard dose forms, having an average weight between 700 mg and 900 mg, preferably between 700 and 800 mg.

It should be noted that according to a preferred embodiment, the composition may be made in oral solid dosage forms selected from tablets, capsules, or powders, preferably capsules.

The tablet may, if necessary, be made with surface incisions such that it is suitable for splitting.

The daily dosage of at least 400 mg/day of tocotrienol can advantageously be achieved with two administrations per day of the aforementioned oral solid dosage forms.

It should be noted that the lyophilisation process allows to obtain an Annatto powder which, at the same volume or weight, contains a higher tocotrienol content. In particular, the lyophilised Annatto powder has a bulk density of >0.68 g/cm³, preferably 0.68-0.78 g/cm³. This means that, in order to achieve the effective dosage in tocotrienols, a lower amount by weight and a reduced volume of lyophilised Annatto powder is necessary, compared to that required if the pre-lyophilisation powder must be used.

Test Data

Effect of administration of Tocotrienol of Annatto at 400 mg/day in patients with preoperative non-metastatic breast cancer (for at least four weeks from diagnosis to surgery).

The Applicants activated c/o the National Cancer Institute of Milan—IRCCS Foundation—a research protocol aimed at women diagnosed with breast cancer, integrating the use of a product with a high concentration of tocotrienols, and evaluated the effect of oral administration of tocotrienols (in particular of Annatto delta-tocotrienols) at a dose of 400 mg/day, derived from Annatto Bixa Orellana L.

The administration of tocotrienol began at the time of definitive diagnosis of carcinoma and lasted for four weeks until surgery.

The primary end point was to evaluate the anti-inflammatory and antioxidant effect on plasma samples taken from the time of diagnosis and after four weeks of use of tocotrienol.

The study enrolled 50 patients with breast cancer in stage T1-2, NO-1, MO who received a 400 mg/day dose of Tocotrienol in the pre-surgical stage.

In 10 patients, the T mediated and specific immunosuppressive anti-tumour immune response (involving regulatory T cells and suppressive myeloids) in peripheral blood was assessed.

The plasma sample analyses showed a significant reduction in the oxidising effect (tock fast-Li Starfish) and VEGF (vascular endothelial growth factor) at the end of the treatment, compared to the baseline values. The antioxidant activity (TAC Track, Li Starfish) was significantly increased after the treatment.

A modification of the T-mediated anti-tumour immune response and specific immunosuppression status was documented: in particular, for both the inflammatory myeloid line and the granulocytic and monocytic immunosuppressive line, a significant reduction was recorded at the end of the treatment, together with the regulatory T cells. The preliminary data showed an effect on cytolytic NK cells and Th1 CD4+ activated T-helper cells, which were significantly increased.

These results showed that the Annatto delta-tocotrienol had an antioxidant and anti-inflammatory effect but also, in the immunological profile, a selective antagonistic effect in the neoplastic sense in this group of patients treated in the pre-operative stage.

Supported by these preliminary data, the analyses were extended not only on plasma markers but also on tissue markers, relating in particular to immune function, and improvements in the biological profile of the primitive tumour were highlighted.

The Nanostring nCounter tool was used for this analysis, which is capable of gene expression analysis starting from formalin-fixed paraffin-embedded samples.

Once the RNA was extracted from the samples, the expression of approximately 770 immune-related genes was analysed (genes related to 24 different immune cells, check-point inhibitor drug target genes, innate and adaptive immunity genes are included in the Pan Cancer Immune Profiling panel).

Lastly, by virtue of the software used, a first statistical analysis of the samples was obtained, highlighting the genes which patients express significantly before and after treatment.

From the preliminary data performed on 12 biopsies (pre-treatment with tocotrienol) and 12 operating pieces (post-treatment with tocotrienol), the genes expressed with significant differences were compared in the pre-treatment phase and their corresponding post-treatment phase for each individual gene identified.

A significant up-regulation of post-treatment genes was identified compared to pre-treatment, and these genes (e.g., EGR1, ERG2) have a potential degenerative effect of cancer cells (stressful cellular effect of apoptosis).

Some down-regulated genes were also observed in post-treatment compared to pre-treatment; for the latter no definitive conclusions can yet be drawn and the role thereof will be clarified in the investigation phase. For the purposes of information and not definitively, it should be noted that the down-regulated genes include for example SMAD2, which is a regulator of multiple cellular processes such as proliferation, apoptosis and angiogenesis and is directly associated with a worse prognosis in breast cancer patients; ENTPD1, expressed by regulatory T cells, which promotes tumour growth.

Our data significantly and innovatively demonstrate the constant unidirectional effect of tocotrienol from Annatto on the genes highlighted in tumour tissues, both repressively and in an amplifying sense.

This effect opens the possibility of a possible clinical finding consistent with the laboratory data.

Our results on patients have so far no precedent in the literature.

These preliminary data on the tissues of patients with non-metastatic breast cancer suggest a possible therapeutic indication of Tocotrienol from Annatto as an adjuvant in the oncological treatment of non-metastatic breast cancer, and not only as a generic antioxidant.

### EXAMPLES

Purely by way of non-limiting illustration, examples of the method and composition object of the present application are given below.

1. Pharmaceutical composition—capsules (volume: 0.95ml, length:23.3mm)

| Ingredients | mg/tab |
|---|---|
| Annatto Bixa Orellana L. Powder (Tocotrienol ≥40%) | 620 |
| Glycine | 15 ÷ 20 |
| Microcrystalline cellulose | 80 ÷ 100 |
| Silicon Dioxide | 1 ÷ 3 |
| Fatty acid Magnesium salts | 1 ÷ 3 |

Controls

| Analysis | | Measurement | Release | | | Not |
|---|---|---|---|---|---|---|
| description | Result | unit | specification | Method | Compliant | compliant |
| Appearance | White/blue | | White/blue | Organoleptic | X | / |
| and colour | capsule | | capsule | | | |
| Format | | mm | | Dimensional | X | / |
| Odour | Odourless | | Odourless | Organoleptic | X | / |
| Weight | | mg | | Internal | X | / |
| | | | | method | | |

2. Basic lyophilisation method—comparison example

The present example describes one of the lyophilisation methods performed by the Applicants as the first attempt to treat Annatto powder. The present method obtained a powder unsatisfactory for the purposes of the invention; in particular, the powder was sticky, non-homogeneous, with an excessive apparent volume.

For the purposes of the present method, seeds of Annatto Bixa Orellana L. were prepared by grinding or crushing, or lyophilised as is without any pre-treatment.

| | Temperature | Pressure | | |
|---|---|---|---|---|
| Process | (° C.) | (mbar) | Time | |
| Pre-freezing | −15 | | 9 | h |
| Main freeze-drying | −15 | 1.2 | 30 | min |
| | −12 | 1.2 | 4 | h |
| | −8 | 2.0 | 4 | h |
| | −4 | 2.5 | 2 | h |
| Secondary freeze-drying | 0 | 1.000 | 1 | h |
| | 5 | 1.000 | 2 | h |
| | 10 | 1.000 | 2 | h |
| | 15 | 1.000 | 4 | h |
| | 20 | 1.000 | 4 | h |

3. Annatto powder lyophilisation method—according to the invention

The product was prepared by grinding in a mixer to homogenise the product as much as possible.

| | Temperature | | | |
|---|---|---|---|---|
| Process | (° C.) | Pressure | Time | |
| Pre-freezing | −40 | | 18 | h |
| Main freeze-drying | −45 | 0.2 | 30 | min |
| | −40 | 0.2 | 4 | h |
| | −35 | 0.2 | 4 | h |
| | −30 | 0.5 | 4 | h |
| | −25 | 0.5 | 4 | h |
| | −20 | 0.7 | 4 | h |
| | −15 | 0.7 | 4 | h |
| | −10 | 1.2 | 4 | h |
| | −5 | 1.2 | 4 | h |
| | 0 | 1.2 | 4 | h |
| Secondary freeze-drying | 0 | 1.000 | 1 | h |
| | 5 | 1.000 | 2 | h |
| | 10 | 1.000 | 2 | h |
| | 15 | 1.000 | 4 | h |
| | 20 | 1.000 | 4 | h |

4. Weight loss of the lyophilised Annatto powder.

An Annatto seed powder (Bixa orellana L.) with a 35% tocotrienol content was subjected to the lyophilisation process according to the invention.

The raw material specifications are as follows:

PRODUCT DATA

| | |
|---|---|
| Product name: | ANNATTO TOCOTRIENOLS |
| | DELTAGOLD 35 P |
| Botanical name: | Bixa Orellana L. |
| Part of the plant: | seed |
| Support: | tapioca dextrin |
| Origin: | America |
| Appearance | Powder |
| Colour | Yellow-gold |
| Total tocotrienols (T3) | >=35% (350 mg/g) |
| Delta-tocotrienols | 84-92% of total T3 |
| Gamma-tocotrienols | 8-16% of total T3 |
| Other tocotrienols and | <1% |
| tocopherols | |
| Humidity | <5% |
| Peroxide index | <5 meq/Kg |
| Heavy metals | <10 ppm |
| As | <2 ppm |
| Total bacterial count | <1000 cfu/g |
| Moulds and yeasts | <100 cfu/g |
| Coliform Pathogens: | <10 cfu/g |
| E. Coli: | Absent |
| Salmonella species: | absent |
| Validity | 24 months from date of manufacture in |
| | original packaging |
| Conservation | In a cool, dry place in original |
| | containers. Store the product away from |
| | light and moisture (10° C.-20° C.) |

The powder was subjected to the lyophilisation process according to the invention.

Lyophilisation parameters

| Raw material weight | 500.4 g |
|---|---|
| Lyophilised product weight | 493.7 |
| Yield % | 98.66% |

It should be noted that the lyophilised powder had a weight reduction, attributable to water loss, of 1.34%.

In relation to the water content assumed on the total raw material (25.02 g), with a weight loss of 6.7 g, it is estimated that the water loss percentage was about 26.77%; the residual water content in the final powder is estimated to be 3.7% (about 18.32 g).

1. Lyophilisation process of Annatto Bixa Orellana L. powder comprising the steps of:
a) providing Annatto seed powder with a tocotrienol content >35% (w/w);
b) freezing Annatto powder at a temperature of <-15° C.;
c) submitting the frozen powder according to phase (b) to a freeze-drying process characterized in that the freeze-drying step (c) comprises the sub-steps of
i) performing a first drying step at a pressure of <2.5 mbar and a temperature between
−45° C. and 0° C.;
ii) performing a second drying step, following the first drying step (i), at a constant pressure greater than the pressure of the first drying, at a temperature between 0° C. and 20° C.,
wherein the Annatto powder resulting from the freeze-drying phase (c) has a tocotrienol content ≥39% (w/w).

2. Process according to claim 1, in which the first drying step lasts more than 30 hours.

3. Process according to claim 1 in which the second drying step lasts at least 12 hours.

4. Process according to claim 1, wherein the Annatto seed powder is provided, in step (a), in combination with a cryo-preservative.

5. Process according to claim 1, wherein the Annatto powder resulting from the freeze-drying phase (c) has a residual water content ≤4,00% (w/w).

6. Pharmaceutical composition comprising lyophilised powder of Annatto Bixa Orellana L. obtained with the process according to claim 1, in combination with suitable excipients and/or diluents, wherein the Annatto powder has a tocotrienol content ≥39% (w/w), a residual water content ≤4,00% (w/w).

7. Pharmaceutical composition according to claim 6, comprising Annatto powder in amounts ranging between 60% and 90% (w/w).

8. Pharmaceutical composition according to claim 7, further comprising a cryo-preservative in amounts ranging between 0.5% and 5% (w/w).

9. Pharmaceutical composition according to claim 6 in form of tablets, capsules or powder.

10-14. (canceled)

15. Process according to claim 5, wherein the Annatto powder resulting from the freeze-drying phase (c) has an apparent volume ≥0,68 g/cm³.

16. Pharmaceutical composition comprising lyophilised powder of Annatto Bixa Orellana L. obtained with the process according to claim 6, wherein the Annatto powder has an apparent volume ≥0,68 g/cm³.

17. Pharmaceutical composition according to claim 6, in form of oral solid dosage form.

18. Pharmaceutical composition according to claim 17, wherein each solid dosage form has an average weight between 700 mg and 900 mg.

19. Pharmaceutical composition according to claim 18, wherein a daily dosage of 400 mg of tocotrienol is delivered within a maximum of two oral solid dosage form.

20. A method for treating non-metastatic breast cancer during the pre-operative stage of an oncological treatment, said method comprising
orally administering a therapeutically effective amount of a composition comprising lyophilised powder of Annatto Bixa Orellana L. in combination with suitable excipients and/or diluents, wherein the Annatto powder has a tocotrienol content ≥39% (w/w), a residual water content ≤4,00% (w/w), and an apparent volume ≥0,68 g/cm³.

21. The method according to claim 20, wherein said composition is administered in addition to systemic or regional adjuvant treatment, or in addition to systemic or regional neo-adjuvant treatment.

22. The method according to claim 20, wherein a daily dosage of 400 mg of tocotrienol is delivered within a maximum of two oral solid dosage form.
